(19) United States
(12) Patent Application Publication
(10) Pub. No.: US 2016/0200667 A1
(43) Pub. Date: Jul. 14, 2016

(54) NOVEL PROCESS FOR MAKING OMEGA-AMINOALKYLENIC ALKYL ESTER

(71) Applicant: INVISTA NORTH AMERICA S.A R.L., WILMINGTON, DE (US)

(72) Inventors: Ronald L. AMEY, Wilmington, DE (US); Anne GAFFNEY, West Chester, PA (US); Frank E. HERKES, Wilmington, DE (US)

(73) Assignee: INVISTA NORTH AMERICA S.A R.L., WILMINGTON, DE (US)

(21) Appl. No.: 14/990,852

(22) Filed: Jan. 8, 2016

Related U.S. Application Data
(60) Provisional application No. 62/103,131, filed on Jan. 14, 2015.

(51) Int. Cl. C07C 229/30; C07C 69/533

(52) U.S. Cl. CPC C07C 229/30; C07C 69/533; C07C 2101/20

(57) ABSTRACT

A method for making a compound of formula (V):

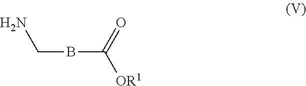

is provided. The method comprises converting a compound of formula (III):

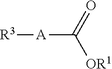

to the compound of formula (V), wherein A is a C₆-C₁₀ alkene group having at least one carbon-carbon double bond, B is a C₆-C₁₀ alkyl chain; and R¹ is an alkyl group, and R³ is an oxygenated functional group.

### FIELD OF THE INVENTION

The disclosures herein relate to a method for making a compound of formula (V):

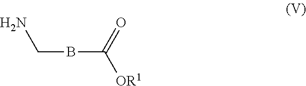

comprising converting a compound of formula (III):

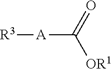

to the compound of formula (V), wherein A is a C₆-C₁₀ alkene group having at least one carbon-carbon double bond, B is a C₆-C₁₀ alkyl chain; and R¹ is an alkyl group, and R³ is an oxygenated functional group.

### BACKGROUND OF THE INVENTION

U.S. Pat. No. 5,530,148 discloses a process by which vernolic acid (cis-12,13-epoxy-cis-9-octadecenoic) is transformed into 12-oxododecanoic acid oxime, which can be further transformed into 12-aminododecanoic acid, the monomer for Nylon-12.

U.S. patent application publication No. 2013/0116458 A1 discloses a process for the synthesis of long-chain α,ω-aminoalkanoic acids or esters from a monounsaturated fatty acid or ester comprising at least one metathesis stage.

U.S. Pat. No. 3,483,253 discloses a process for the production of amines by reacting an aldehyde or ketone with liquid ammonia and hydrogen at a temperature from 90 to 130° C. under a pressure of from 60 to 700 atmospheres gauge in the presence of a catalyst which contains nickel and/or cobalt, chromium and at least one non-volatile mineral acid which is capable of being converted into an insoluble anhydride or an insoluble polyacid.

U.S. Pat. No. 3,933,873 discloses a method for preparing omega-aminoalkanoic acids by the steps of nitro-oxidizing a cycloalkene to a cyclic alpha-nitroketone, cleaving and esterifying a cyclic alpha-nitroketone with an alcohol to form an alkyl omega-nitroester, catalytically hydrogenating the nitroester to an aminoester and hydrolyzing the aminoester to an aminoalkanoic acid.

GB Patent No. 1,514,402 discloses a process for preparing saturated omega-aminoacids from olefinically unsaturated omega-aldehydoacids.

U.S. Pat. No. 4,329,297 discloses a process for preparing, in a single hydrogenation step, saturated omega-amino acids from olefinically unsaturated omega-aldehyde acids.

U.S. Pat. No. 3,883,586 discloses a process for preparing the C₁-C₄ alkyl esters of omega-aminododecanoic acid by catalytic hydrogenation of omega-nitro-C₁₂-carboxylic acid esters in the presence of a hydrocarbon solvent.

U.S. Pat. No. 4,218,384 (the '384 patent) discloses a process of making an omega-amino alkenoic acid containing from 8 to 12 carbon atoms from an omega-formyl alkenoic acid, also containing from 8 to 12 carbon atoms.

There are several disadvantages to the process given in the '384 patent, namely:
(1) The occurrence of carboxylic acid end group functionality in the substrate presents a real problem during the conversion of aldehyde end group to amine end group; and
(2) It is necessary to shield the carboxylic acid end group via saltification followed by its recovery via acidification (reversal of saltification) steps that involve environmentally unfriendly alkali metal salts and strong acids.

For the above facts, the '384 patent required a special sequence of 1) ammonia addition, to first iminate the aldehyde end group, 2) followed by saltification to shield the carboxylic acid end group, 3) followed by reduction of the imine carboxylate salt for converting the imine end group to its corresponding amine end group while subsequently reducing all carbon-carbon double bonds, and finally, 4) acidification to convert the salt back to the carboxylic acid functionality. The '384 patent obtained the intended omega-amino acid using this order of steps.

As a result, the '384 patent preparation is complex and involves environmentally unfriendly alkali metal salts and strong acids following the saltification step. Thus, there is still a need to develop a simplified and improved process for making omega-amino alkenoic alkyl esters.

### SUMMARY OF THE INVENTION

One aspect of the disclosed process is directed to a method for making a compound of formula (V):

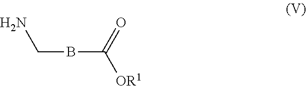

comprising converting a compound of formula (III):

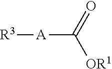

to the compound of formula (V), wherein A is a C₆-C₁₀ alkene group having at least one carbon-carbon double bond, B is a C₆-C₁₀ alkyl chain; and R¹ is an alkyl group, and R³ is an oxygenated functional group.

Another aspect of the disclosed process is directed to a method for making a compound of formula (V):

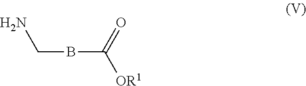

comprising
a. contacting a compound of formula (I):

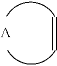

and a reagent with a medium comprising ozone to form a compound of formula (IIa):

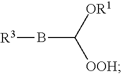

b. allowing the compound of formula (IIa) transform to a compound of formula (III):

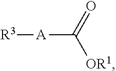

without forming an acetate side product; and
c. converting the compound of formula (III) to the compound of formula (V), wherein A is a C₆-C₁₀ alkene group having at least one carbon-carbon double bond, B is a C₆-C₁₀ alkyl chain; and R¹ is an alkyl group, and R³ is an oxygenated functional group.

Another aspect of the disclosed process is directed to a method for making a compound of formula (V):

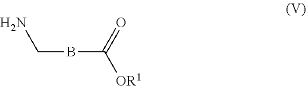

comprising
a. contacting a compound of formula (I′):

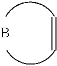

and a reagent with a medium comprising ozone to form a compound of formula (IIa′):

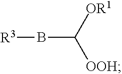

b. allowing the compound of formula (IIa′) transform to a compound of formula (IV):

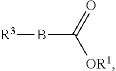

without forming an acetate side product; and
c. converting the compound of formula (IV) to the compound of formula (V), wherein B is a C₆-C₁₀ alkyl chain; and R¹ is an alkyl group, and R³ is an oxygenated functional group.

Another aspect of the disclosed process is directed to a composition of matter comprising a compound of formula (IX):

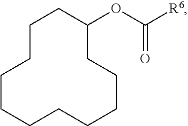

wherein R⁶ is C₄-C₂₀ alkyl or benzyl.

Another aspect of the disclosed process is directed to a composition of matter comprising a compound of formula (X):

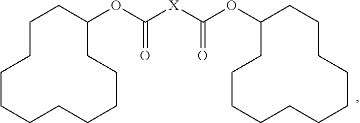

wherein X is C₁-C₁₂ alkyl, phenyl group with optional substitution, or C₁-C₄ alkyl-phenyl-C₁-C₄ alkyl.

### BRIEF DESCRIPTION OF THE DRAWING

FIG. 1 is a representation of an embodiment of an overall reaction scheme in the present invention.

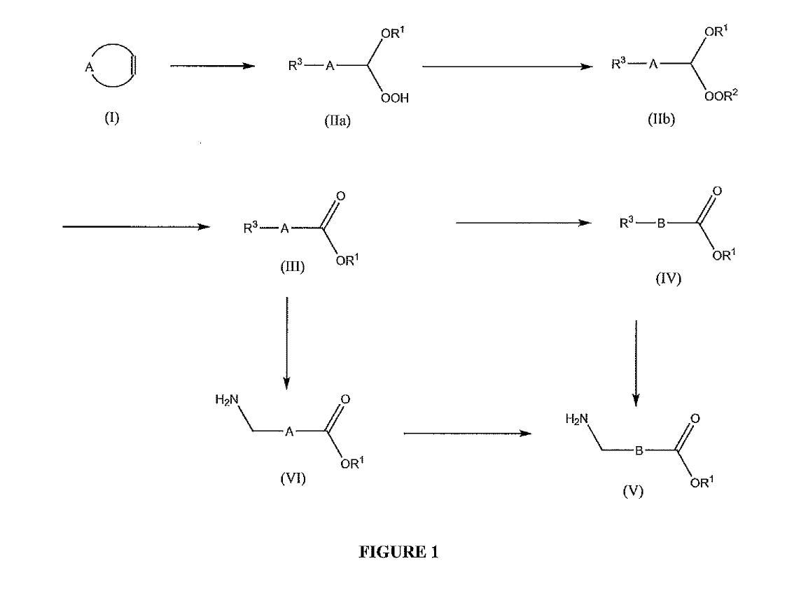

FIG. 2 is a representation of an embodiment of the method of preparations involving the sequence as required herein.

FIG. 3 is a representation of an embodiment according to Examples 16 through 25.

FIG. 4 is a representation of an embodiment according to Examples 16 through 25.

### DETAILED DESCRIPTION OF THE INVENTION

Embodiments of the invention described and claimed herein are not to be limited in scope by the specific embodiments herein disclosed, since these embodiments are intended as illustration of several aspects of the disclosure. Any equivalent embodiments are intended to be within the scope of this disclosure. Indeed, various modifications of the embodiments in addition to those shown and described herein will become apparent to those skilled in the art from the foregoing description. Such modifications are also intended to fall within the scope of the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

As used herein and in the appended claims, the singular forms “a,” “an,” and “the” include plural reference unless the context clearly dictates otherwise. Thus, for example, a reference to “a reactor” includes a plurality of reactors, such as in a series of reactors. In this document, the term “or” is used to refer to a nonexclusive or, such that “A or B” includes “A but not B,” “B but not A,” and “A and B,” unless otherwise indicated.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about (X) to Y” has the same meaning as “about (X) to about Y,” unless indicated otherwise. Likewise, the statement “about X, Y, or about Z” has the same meaning as “about X, about Y, or about Z,” unless indicated otherwise.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing (X) and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “alkene” as used herein refers to a linear or branched hydrocarbon olefin that has at least one carbon-carbon double bond.

The term “alkyl” or “alkylene” as used herein refers to a saturated hydrocarbon group which can be an acyclic or a cyclic group, and/or can be linear or branched unless otherwise specified.

Herein, the term “ozonolysis product” is meant to include those structures, transient or otherwise susceptible to isolation if desired, resulting from the reaction of one ozone molecule with a single double bond of the compound of formula I, wherein the attacked double bond is not in group A of the compound of formula I. However, the applicants do not wish to be limited by or subject to any particular mechanistic interpretation as to the formation of an ozonolysis product.

The term “reagent” as used herein means a consumable material that provides the suitable R¹ functionality. In some embodiments, the reagent is polar. In other embodiments, the reagent provides a single continuous phase of the reaction. In yet another embodiment, the reagent improves the flowability characteristics of the reaction medium. In some embodiments, the reagent improves the heat transfer properties of the reaction medium.

All publications, including non-patent literature (e.g., scientific journal articles), patent application publications, and patents mentioned in this specification are incorporated by reference as if each were specifically and individually indicated to be incorporated by reference.

It is understood that the descriptions herein are intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” “third,” and the like are used merely as labels, and are not intended to impose numerical requirements on their objects.

In an embodiment, conversion (mass or molar) defined as a percent as follows:

$$\frac{[ \begin{matrix}( \begin{matrix}Number of grams or moles of \\ compound present prior to \\ chemical reaction step\end{matrix} ) - \\ ( \begin{matrix}Number of grams or moles of \\ compound present after \\ chemical reaction step\end{matrix} )\end{matrix} ]}{( \begin{matrix}Number of grams or moles of \\ compound present prior to \\ chemical reaction step\end{matrix} )} \times 100$$

In an embodiment, a product yield (mass or molar) defined as a percent as follows:

$$\frac{[ \begin{matrix}( \begin{matrix}Number of grams or moles of \\ compound present after \\ chemical reaction step\end{matrix} ) - \\ ( \begin{matrix}Number of grams or moles of \\ product present prior to \\ chemical reaction step\end{matrix} )\end{matrix} ]}{( \begin{matrix}Number of grams or moles of \\ compound consumed during \\ chemical reaction step\end{matrix} )} \times 100$$

One aspect of the disclosed process is directed to a method for making a compound of formula (V):

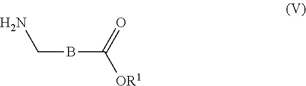

comprising converting a compound of formula (III):

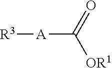

to the compound of formula (V), wherein A is a C₆-C₁₀ alkene group having at least one carbon-carbon double bond, B is a C₆-C₁₀ alkylene chain; and R¹ is an alkyl group, and R³ is an oxygenated functional group.

In some embodiments, R¹ is a C₁-C₆ alkyl. In a further embodiment, R¹ is a C₁-C₅ alkyl. In another embodiment, R¹ is a C₂-C₄ alkyl. In a further embodiment, R¹ is propyl or butyl.

In some embodiments, A is a C₆ or C₁₀ alkene chain with at least one double bond. In one embodiment, A is a C₁₀ alkene with two double bonds. In another embodiment, A is a C₆ alkene with one double bond.

In some embodiments, R³ is an aldehyde, an acid, or an ester group. In a further embodiment, R³ is an aldehyde or an acid group. In another further embodiment, R³ is an aldehyde group.

In some embodiments, B is a C₆ or C₁₀ alkylene chain. In one embodiment, B is a C₁₀ alkylene. In another embodiment, B is a C₆ alkylene.

In some embodiments, the compound of formula (III) is first converted to a compound of formula (IV):

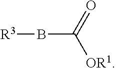

In some embodiments, the conversion to the compound of formula (IV) is conducted under a suitable hydrogenation condition. Suitable hydrogenation condition may employ a catalyst and may be carried out in any synthetically feasible manner to effectively provide the compound of formula (IV) in suitable yield and/or purity. The reagents, catalysts, solvents and/or reaction conditions can be appropriately selected. The hydrogenation condition may typically include contact time, temperature, pressure, type of catalyst, catalyst concentration, and concentration of the starting material.

Suitable hydrogenation condition and catalysts are disclosed, e.g. in Compendium of Organic Synthetic Methods (John Wiley & Sons, New York) Vol. 1, Ian T. Harrison and Shuyen Harrison (1971); Vol. 2, Ian T. Harrison and Shuyen Harrison (1974); Vol. 3, Louis S. Hegedus and Leroy Wade (1977); Vol. 4, Leroy G. Wade Jr., (1980); Vol. 5, Leroy G. Wade Jr. (1984); and Vol. 6, Michael B. Smith; as well as March, J., Advanced Organic Chemistry, 3rd Edition, John Wiley & Sons, New York (1985); Comprehensive Organic Synthesis. Selectivity, Strategy & Efficiency in Modern Organic Chemistry, In 9 Volumes, Barry M. Trost, Editor-in-Chief, Pergamon Press, New York (1993); Advanced Organic Chemistry, Part B: Reactions and Synthesis, 4th Ed.; Carey and Sundberg; Kluwer Academic/Plenum Publishers: New York (2001); Advanced Organic Chemistry, Reactions, Mechanisms, and Structure, 2nd Edition, March, McGraw Hill (1977); and Comprehensive Organic Transformations, 2nd Edition, Larock, R. C., John Wiley & Sons, New York (1999).

In some embodiments, the hydrogenation catalyst may comprise nickel (Ni), cobalt (Co), chromium (Cr), iron (Fe), a precious metal catalyst, such as palladium (Pd), platinum (Pt), or ruthenium (Ru); or a combination thereof. The palladium metal catalyst is preferred.

In some embodiments, metals may be supported on carriers such as ceria, alumina, titania, silica, carbon, silica-aluminas, mixed metal oxides, zeolites, mesoporous materials, micro porous materials, structured materials such as monoliths, foams, and the like. The metals supported on silica, alumina or carbon supports are preferred; the metals supported on silica or carbon being most preferred.

In some embodiments, the suitable hydrogenation condition may comprise a hydrogenation agent. In other embodiments, the hydrogenation agent may be a hydrogen-containing medium. In one embodiment, the hydrogenation agent may be a hydrogen gas. The hydrogen gas of desired purity may be obtained from any of the commercial, industrial, bio-facility, electrolysis, and gas separation membrane installations.

In some embodiments, the suitable hydrogenation condition may employ a hydrogenation agent, a solvent and a catalyst, at an elevated pressure and an elevated temperature. In one embodiment, the hydrogenation employs hydrogen gas (H₂), a catalyst, and a solvent. In another embodiment, the hydrogenation employs hydrogen gas (H₂), catalyst, and ammonia.

In some embodiments, the hydrogenation is carried out at a temperature of above 0° C., e.g., above 10° C. In other embodiments, the hydrogenation is carried out at a temperature of up to 150° C. In some other embodiments, the hydrogenation is carried out at a temperature of about 0° C. to about 150° C., e.g. from about 10° C. to about 125° C., from about 10° C. to about 100° C., from about 10° C. to about 80° C., or from about 10° C. to about 75° C.

In some embodiments, the hydrogenation is carried out at an elevated pressure (e.g., above 1 atm). In other embodiments, the hydrogenation is carried out at a pressure of up to 100 atm, e.g. up to 75 atm, up to 60 atm.

In some embodiment, the molar ratio range of hydrogen and the compound of formula (III) is from about 1:10 to about 50:1, preferably between about 1:5 and about 20:1.

In some embodiments, the conversion from the compound of formula (IV) to the compound of formula (V) is conducted under a suitable reductive amination condition. Suitable reductive amination condition may employ a catalyst and may be carried out in any synthetically feasible manner to effectively provide the compound of formula (V) in suitable yield and/or purity. The reagents, catalysts, solvents and/or reaction conditions can be appropriately selected. The reductive amination condition may typically include contact time, temperature, pressure, concentration of reductant, type of catalyst with active metal loading, catalyst concentration, and concentration of the starting material.

In some embodiments, the reductive amination condition may employ any of the cobalt (Co), nickel (Ni), chromium (Cr), iron (Fe), molybdenum (Mo), platinum (Pt), palladium (Pd), Raney® nickel, Raney® cobalt catalyst, or combination thereof. In other embodiments, the reductive amination condition may employ any of the activated metal or skeletal catalysts.

In some embodiments, the suitable reductive amination condition may comprise a reductive amination agent. In other embodiments, the reductive amination agent may be an ammonia-containing medium. In one embodiment, the reductive amination agent may be an aqueous solution of ammonia-containing medium, for example, an aqueous solution of ammonium hydroxide (NH₄OH). In another embodiment, the reductive amination agent may be an ammonia gas. The ammonia gas of desired purity may be obtained from any of the commercial, industrial, bio-facility, electrolysis, and gas separation membrane installations.

In some embodiments, the molar ratio range of ammonia and the compound of formula (IV) is from about 1:10 to about 50:1, preferably between about 1:5 and about 30:1, more preferably between about 1:4 and about 20:1.

In some embodiments, the reductive amination is carried out at a temperature of above 0° C., e.g., above 10° C. In other embodiments, the reductive amination is carried out at a temperature of up to 150° C. In some other embodiments, the reductive amination is carried out at a temperature of about 0° C. to about 150° C., e.g. from about 10° C. to about 125° C., from about 10° C. to about 100° C., from about 10° C. to about 80° C., or from about 10° C. to about 75° C.

In some embodiments, the reductive amination is carried out at an elevated pressure (e.g., above 1 atm). In other embodiments, the reductive amination is carried out at a pressure of up to 100 atm, e.g. up to 75 atm, up to 60 atm.

Readers may appreciate the fact that there is no limitation on how much excess hydrogen and/or ammonia one can feed except the unit productivity will be adversely impacted by a large excess. Also, the downstream equipment may have to be oversized to handle such large excess. An acceptable balance between the productivity and equipment sizes may be required from economical and operational standpoints.

In some embodiments, a continuous mode reactor may be used with a heterogeneous or homogeneous catalyst. In other embodiments, the reactor may be a fixed bed, continuous stirred tank reactor (CSTR), slurry bed, bubble column, trickle bed, microstructure, membrane or other possible configurations. In one embodiment, the gas and/or liquid flows may be co-current or counter-current. In some other embodiments, the direction of the gas and/or liquid flows may be up-flow, down-flow or of the cross-flow type. In another embodiment, the gases and/or hydrocarbon feed may be added in stages and distributed uniformly either radially, axially, perpendicular to the flow or in the direction of the flow. In yet another embodiment, multiple series of reactor systems may be employed.

Suitable exemplary solvents useful for the hydrogenation and reductive amination include, alcohols (e.g. methanol, ethanol, 1-propanol, isopropanol, 2-methyl-2-propanol, 1-butanol, 2-butanol, isobutanol, 2-methyl-2-butanol, tert-butanol, 3-pentanol), amides such as dimethylformamide (DMF) and dimethylacetamide (DMAc), dimethyl sulfoxide (DMSO), hydrocarbons (e.g. hexane, pentane, benzene, xylene or toluene), tetrahydrofuran (THF), dioxane, and N-methyl-2-pyrrolidone (NMP).

In other embodiments, the compound of formula (III) is first converted to a compound of formula (VI):

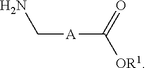

In some embodiments, the conversion to the compound of formula (VI) is conducted under a suitable reductive amination condition.

In one embodiment, the suitable reductive amination condition comprising a reductive amination agent.

Examples of suitable reductive amination agent include, but are not limited to, ammonia.

In some embodiment, the molar ratio range of ammonia and the compound of formula (III) is from about 1:10 to about 50:1, from about 1:4 to about 20:1, or from about 1:2 to about 10:1.

In some embodiments, the conversion from the compound of formula (VI) to the compound of formula (V) is conducted under a suitable hydrogenation condition. In one embodiment, the suitable hydrogenation condition comprises a hydrogenation agent.

Examples of suitable hydrogenation agent include, but are not limited, hydrogen.

In some embodiment, the molar ratio range of hydrogen and the compound of formula (VI) is from about 1:10 to about 50:1, from about 1:5 to about 20:1, or from about 1:3 to about 10:1.

Another aspect of the disclosed process is directed to a method for making a compound of formula (V):

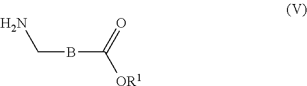

comprising
a. contacting a compound of formula (I):

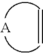

and a reagent with a medium comprising ozone to form a compound of formula (IIa):

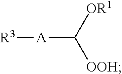

b. allowing the compound of formula (IIa) transform to a compound of formula (III):

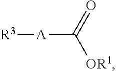

without forming an acetate side product; and
c. converting the compound of formula (III) to the compound of formula (V), wherein A is a C₆-C₁₀ alkene group having at least one carbon-carbon double bond, B is a C₆-C₁₀ alkylene chain; and R¹ is an alkyl group, and R³ is an oxygenated functional group.

In some embodiments, R¹ is a C₁-C₆ alkyl. In a further embodiment, R¹ is a C₁-C₅ alkyl. In another embodiment, R¹ is a C₂-C₄ alkyl. In a further embodiment, R¹ is propyl or butyl.

In some embodiments, A is a C₆ or C₁₀ alkene chain with at least one double bond. In one embodiment, A is a C₁₀ alkene with two double bonds. In another embodiment, A is a C₆ alkene with one double bond.

In some embodiments, R³ is an aldehyde, an acid, or an ester group. In a further embodiment, R³ is an aldehyde or an acid group. In another further embodiment, R³ is an aldehyde group.

In some embodiments, B is a C₆ or C₁₀ alkylene chain. In one embodiment, B is a C₁₀ alkylene. In another embodiment, B is a C₆ alkylene.

In some embodiments, the compound of formula (III) is first converted to a compound of formula (IV):

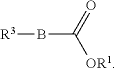

In some embodiments, the conversion to the compound of formula (IV) is conducted under a suitable hydrogenation condition. In one embodiment, the suitable hydrogenation condition comprises a hydrogenation agent.

Examples of suitable hydrogenation agent include, but are not limited, hydrogen.

In some embodiment, the molar ratio range of hydrogen and the compound of formula (III) is from about 1:10 to about 50:1, from about 1:5 to about 20:1, or from about 1:3 to about 10:1.

In other embodiments, the compound of formula (III) is first converted to a compound of formula (VI):

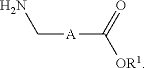

In some embodiments, the conversion from the compound of formula (IV) to the compound of formula (V) is conducted under a suitable reductive amination condition.

In one embodiment, the suitable reductive amination condition comprising a reductive amination agent.

Examples of suitable reductive amination agent include, but are not limited to, ammonia.

In some embodiment, the molar ratio range of ammonia and the compound of formula (IV) is from about 1:10 to about 50:1, from about 1:4 to about 20:1, or from about 1:2 to about 10:1.

In one embodiment, the reagent is provided in excess. In this context, the term “excess” is defined as the molar amount of the reagent is more than the reacted compound of formula I. In some embodiments, the method further comprising at least partially removing the excess reagent prior to b). In a further embodiment, majority of the excess reagent is removed prior to b). In some embodiments, the excess reagent is removed via flash distillation. In another embodiment, the catalyst is at least partially removed with the reagent. In a further embodiment, majority of the catalyst is removed. In some embodiments, the removed catalyst and/or reagent are recycled back to the process.

In some embodiments, the transformation is in the presence of a catalyst.

### Ozonolysis

The compound of formula (IIa) may be obtained by ozonolysis of the compound of formula (I). International Application No. PCT/US2014/045808, filed Jul. 8, 2014, (the '808 application) discloses a process for obtaining the compound of formula (IIa) from a selective ozonolysis of the compound of formula (I). The entire contents and disclosure of the '808 application are incorporated herein by reference.

The compound of formula (I) may include cyclic trienes and cyclic dienes. Examples of the compound of formula (I) include, but are not limited to, cyclohexadiene, cycloheptadiene, cyclooctadiene, cyclooctatetraene, cyclododecadiene, cyclododectriene, cyclododecapentaene including isomers and mixtures thereof. In some embodiments, the compound of formula (I) is cyclododecatriene or cyclooctadiene. In a further embodiment, the compound of formula (I) is 1,5,9-cyclododectriene (CDDT) or 1,5-cyclooctadiene (COD). In another further embodiment, the compound of formula (I) is CDDT.

In some embodiments, the compound of formula (I) has a purity of at least 90%. In other embodiments, the compound of formula (I) has a purity of at least 95%. In a further embodiment, the compound of formula (I) has a purity of at least 98%. In another further embodiment, the compound of formula (I) has a purity of at least 99%.

In some embodiments, the reagent is a C₁-C₁₀ alcohol. Examples of the suitable alcohol include, but are not limited to, methanol, ethanol, 1-propanol, 2-propanol, 1-butanol, 2-butanol, iso-butanol, t-butanol, and mixtures thereof. In some embodiments, the alcohol is 1-propanol, 2-propanol, 1-butanol, 2-butanol, iso-butanol, t-butanol, and mixtures thereof. In other embodiments, the reagent is a C₄-C₁₀ alcohol. Higher alcohols such as butanols, etc., are preferred.

In some embodiments, the reagent is anhydrous, preferably contains less than 0.5 wt. % water, or more preferably less than 0.1 wt. % water. In other embodiments, the water content may be no more than 0.08 wt %, preferably no more than 0.04 wt %.

In some embodiments, the amount of the reagent may vary and generally excess reagent may be used. For purposes of the disclosed process, the molar ratio of the compound of formula (I) to the reagent may be about 100:1 to about 1:100, preferably about 25:1 to about 1:25, and more preferably about 10:1 to about 1:10. In one embodiment, the molar ratio of the compound of formula (I) to the reagent is about 4:1 to about 1:10. In another embodiment, the molar ratio of the compound of formula (I) to the reagent is about 6:1 to about 1:6. In yet another embodiment, the molar ratio of the compound of formula (I) to the reagent is about 3:1 to about 1:3.

In some embodiments, the concentration of the compound of formula (I) in the reaction zone, by weight, may be about 0.1% to 99.9% range. In one embodiment, the compound of formula (I) is present in the about 0.5%-25% concentration range. In other embodiments, the compound of formula (I) is present in the about 25%-35%, in the about 35%-45%, in the about 45%-55%, in the about 55%-65%, in the about 65%-75%, in the about 75%-85%, in the about 85%-95%, or in the about 95-99.9% concentration range. The compound of formula (I) concentration range may be from about 25% to about 85%, preferably from about 30% to about 75%, and more preferably from about 30% to about 65%. In one embodiment, the compound of formula (I) is from about 35% to about 60% by weight.

In some embodiments, the ozonolysis reaction may be conducted in the presence of an optional inert solvent. In other embodiments, the inert solvent is a polar solvent. Examples of the suitable polar solvent include, but are not limited to, C₁-C₆ alkyl acetates, ethers, DMF, DMAc, DMSO, NMP, THF, and mixtures thereof.

The ozone-containing gas may comprise a mixture of ozone and at least one carrier gas. The amount of ozone may vary. In some embodiments, ozone may be from about 0.01 mol. % to about 100 mol. %. In a further embodiment, ozone may be from about 0.1 mol. % to about 10 mol. %, from about 10 mol. % to about 30 mole. %, from about 30 mol. % to about 50 mole. %, from about 50 mol. % to about 70 mole. %, from about 70 mol. % to about 90 mole. %, or from about 90 mol. % to about 100 mole. %. In other embodiments, ozone may be from about 0.1 mol. % to about 25 mol. %. In a further embodiment, ozone may be from about 1 mol. % to about 20 mol. %. In another further embodiment, ozone may be from about 1 mol. % to about 15 mol. %.

The carrier gas may be selected from the group consisting of nitrogen, argon, carbon dioxide, oxygen, air, and mixtures thereof. In one aspect, the ozone-containing gas may comprise ozone, oxygen, and argon. In one embodiment, the ozone-containing gas may comprise ozone, oxygen, and nitrogen. In another embodiment, the ozone-containing gas may comprise ozone and carbon dioxide. There is no restriction on which carrier is to be used so long as it is chemically compatible with ozone and the carrier itself does not lead to undesirable reactions with the hydrocarbon substrate.

In some embodiments, the ozone may be introduced in the dissolved state using an appropriate solvent. In other embodiments, the concentration of ozone may be enriched by injecting ozone into a pressurized circulation loop.

In some embodiments, the concentration of ozone in the ozone-containing medium, by weight, may be from about 0.01% to about 100%. In other embodiments, the ozone concentration may be from about 0.5% to about 5%, from about 5% to about 25%, from about 25% to about 50%, from about 50% to about 75%, from about 75% to about 100%.

In some embodiments, the total addition of ozone is at least partially influenced by the desired conversion and efficiency of ozone take-up. The ozone-containing gas is passed through the reaction solution for a period of time sufficient to permit selective cleavage of only one double bond. In some embodiments, the period of time may range from about 10 minutes to about 300 minutes. In a further embodiment, the period of time may range from about 30 minutes to about 200 minutes. The total ozone fed to the process can be either sub-stoichiometric, stoichiometric or excess with respect to the one double bond in the compound of formula I that is converted. In one example, the total moles of ozone fed to the process are in the stoichiometric ratio with respect to the moles of one double bond in the compound of formula I that is converted.

The flow rate of ozone fed may depend on the scale of operation and the desired conversion within the reaction time chosen. In some embodiments, the ozone flow rate is in the range from about 0.001 g/min to about 1 g/min. In a further embodiment, the ozone flow rate is in the about 0.005 g/min to about 0.8 g/min range. In another further embodiment, the ozone flow rate is in the about 0.01 g/min to about 0.2 g/min range. In yet another further embodiment, the ozone flow rate is in the about 0.02 g/min to about 0.08 g/min range.

In some embodiments, the conversion of the compound of formula (I) is from about 0% to about 100%. In a further embodiment, the conversion of the compound of formula (I) is from about 10% to about 95%. In other further embodiments, the conversion of the compound of formula (I) is from about 20% to about 90%, from about 30% to about 70%, or from about 30% to about 60%. In other embodiments, the conversion of the compound of formula (I) is at least 20%. In a further embodiment, the conversion is at least 25%.

In some embodiments, the ozonolysis effluent may comprise from about 0 wt. % to about 50 wt. % the compound of formula (I), from about 0 wt. % to about 80 wt. % reagent, from about 0 wt. % to about 50 wt. % the compound of formula (IIa), and up to about 15 wt. % non-selective products. The non-selective products may include compounds having two terminal oxygenated groups, which include dialdehydes, diacids, diesters, acid-esters, aldehyde-acids. In some embodiments, at least some of the non-selective products are saturated, for example, linear C₄ species. In a preferred embodiment, the ozonolysis effluent comprises from about 0 wt. % to 50 wt. % of the compound of formula (I), from about 0 wt. % to about 80 wt. % reagent, from about 0 wt. % to about 50 wt. % the compound of formula (IIa), and up to about 10 wt. % non-selective products. In some embodiments, the ozonolysis effluent is a stable, flowable liquid at ambient conditions.

In some embodiments, the compound of formula (IIa) is formed with a selectivity of at least 50%. In other embodiments, the compound of formula (IIa) is formed with a selectivity of at least 60%. In another embodiment, the compound of formula (IIa) is formed with a selectivity of at least 70%. In other embodiments, the selectivity for the compound of formula (IIa) is at least 80%. In another embodiment, the selectivity for the compound of formula (IIa) is at least 85%. In a further embodiment, the selectivity for the compound of formula (IIa) is at least 90%. In another further embodiment, the selectivity for the compound of formula (IIa) is at least 95%. In some embodiments, the selectivity for the non-selective products is less than 10%. In a further embodiment, the selectivity for the non-selective products is less than 5%.

In some embodiments, the ozonolysis reaction may be conducted at a temperature of less than 50° C., preferably from about −25° C. to about 50° C., more preferably from about 0° C. to about 40° C., and most preferably from about 0° C. to about 25° C. The ozonolysis reaction is exothermic and, in some embodiments, the temperature of the reactor is maintained by a cooling system, such as an active jacketed cooler.

In some embodiments, the ozonolysis reaction may be conducted at a pressure from about 100 torr to about 200 Psig. In other embodiments, the ozonolysis reaction may be conducted at a pressure from about 100 torr to about 100 Psig. In a further embodiment, the ozonolysis reaction may be conducted at a pressure from about 0 Psig to about 50 Psig, preferably from about 0 Psig to about 25 Psig, more preferably from about 0 Psig to about 20 Psig, and most preferably from about 0 Psig to about 10 Psig. In some embodiments, the vacuum operation may be most suitable for removing the reaction heat via evaporative cooling and so long as the reaction performance is not adversely impacted.

In some embodiments, the mixture comprising the compound of formula (III) is substantially free of the acetate side product. In one embodiment, the amount of the acetate side product is less than 2% wt. of the mixture comprising the compound of formula (III). In another embodiment, the amount of the acetate side product is less than 1% wt. of the mixture comprising the compound of formula (III).

In some embodiments, when the concentrated compound of formula (IIa) from CDDT was exposed to heat at 10° C./minute temperature ramp from 20° C. to 220° C. the material exhibited sufficient thermal stability up to about 60° C. above which the DSC data indicated measurable heat flow and mass loss. Due to its thermal stability, the excess reagent may be distilled off at about 50° C. under reduced pressure without any measurable decomposition and thus no measurable yield loss. In an embodiment, the excess reagent may be removed at about 15° C. to about 60° C. temperature range and the pressure range of about 0 torr to about 100 torr. In another embodiment, the excess reagent may be removed at about 25° C. to about 55° C. temperature range and the pressure range of about 0.05 to about 30 torr. The net impact is an improved overall process with a simpler backend separations scheme not having to deal with the azeotropes of alcohols, acetates and acids, the ability to cleanly recycle the catalyst, and recovery of the excess reagent in support of either continuous or batch processing without negative impact on the process. Thus, the ozonolysis products do not need to be separated and isolated from the ozonolysis effluent. This leads to further improved efficiencies in the process.

In one embodiment, the compound of formula (IIa) is formed from ozonolysis of CDDT, wherein A is —CH₂—CH₂—CH═CH—CH₂—CH₂—CH═CH—CH₂—CH₂—. This compound of formula (IIa) is thermally stable.

### Catalytic Transformation

International Application. No. PCT/US2014/065456 (“the '456 application”), filed Nov. 13, 2014, discloses a process for transforming a compound of formula (IIa):

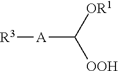

to a compound of formula (III):

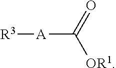

The disclosures of the '456 application are incorporated by reference in their entireties.

In some embodiments, after the excess reagent is removed, the enriched product stream is catalytically transformed in the presence of a catalyst to form the compound of formula (III). In other embodiments, a homogeneous catalyst complex was used for the catalytic transformation reaction. In yet another embodiment, a heterogeneous catalyst may be used for the transformation reaction.

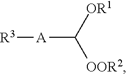

In some embodiments, a compound of formula (IIb): is first formed from the reaction of the compound of formula (IIa) with an anhydride, wherein R² is an acetyl group. The compound of formula (III)) is then contacted with the homogeneous catalytic complex generated “in-situ” from stoichiometrically liberated acid and added amine to be catalytically transformed to the compound of formula (III). The anhydride yields a quantitative conversion of the compound of formula (IIa) to the compound of formula (IIb). Similar to the compound of formula (IIa), the compound of formula (IIb) is also thermally stable.

The catalytic transformation is selective to the peroxy bond and does not react with the double bonds of a compound of formula (IIb):

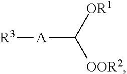

wherein A is a C₆-C₁₀ alkene chain with at least one double bond; R¹ is a C₁-C₁₀ alkyl; R² is H or acetyl; and R³ is an oxygen-containing functional group.

In some embodiments, the anhydride and amine-acid complex are added to a mixture containing the compound of formula (IIa) that is substantially free of the reagent. In another embodiment, the reagent in the mixture is less than 1 wt %. In yet another embodiment, the reagent in the mixture is less than 0.5 wt %.

In some embodiments, the conversion for the catalytic transformation from the compound of formula (IIa) to the compound of formula (III) is between 0 and 100%. In one embodiment, the conversion is in the range of about 0 to about 20%, about 20 to about 40%, about 40% to about 60%, about 60% to about 80%, about 80% to about 100%. In another embodiment, the conversion is at least 90%, preferably at least 95%, and more preferably at least 99%. The catalytic transformation may be conducted at temperatures less than 50° C., preferably range from about 0° C. to about 50° C., and more preferably from about 5° C. to about 40° C.

The catalytic transformation is exothermic and, in some embodiments, the temperature of the reactor is maintained by a cooling system, such as an active jacketed cooler, to maintain a temperature of less than, e.g., 40° C. In some embodiments, the catalytic transformation may be conducted at a pressure from about 0 Psig to about 30 Psig. In other embodiments, the vacuum condition may be suitable when evaporative cooling is used. In a further embodiment, the catalytic transformation may be conducted at a pressure from about 0 Psig to about 5 Psig.

In some embodiments, the catalytic complex is an azeotropic acid-amine complex. This catalytic complex may be recovered from the product mixture via azeotropic distillation and recycled back to the transformation reactor.

With reference to the Schreiber article, the disclosed process takes advantage of the stable azeotropic composition formed between the acid, amine and anhydride such that the equilibrated composition is recovered and recycled into the process. The Schreiber one-pot method is not suitable for commercial manufacturing as it fails to disclose or teach any efficient or economic means for recovering the recyclable components. In contrast, the disclosed process in this invention provides a practical and cost-effective continuous or batch process for commercial production.

The choice of molar addition ratio of the anhydride to amine will depend on the reaction conditions, namely, the contact time, temperature, pressure, heat removal, and such. In one embodiment, the molar ratio of anhydride to amine is from about 1:50 to about 50:1. In another embodiment, the molar ratio of anhydride to amine is from about 1:20 to about 20:1. In yet another embodiment, the molar ratio of anhydride to amine is from about 1:10 to about 10:1.

In some embodiments, the molar feed ratio of the amine to the compound of formula (I) is from about 1:100 to about 10:1, preferably from about 1:25 to about 5:1, more preferably from about 1:20 to about 2:1. In other embodiments, the molar feed ratio of the amine to the compound of formula (I) is from about 1:10 to about 2:1. In another embodiment, the molar feed ratio of the amine to the compound of formula I is from about 1:2 to about 2:1

In some embodiments, the molar feed ratio of the anhydride to the compound of formula (I) may be from about 1:100 to about 100:1. In other embodiments, the molar feed ratio of the anhydride to the compound of formula (I) is from about 1:25 to about 25:1. In one embodiment, the molar feed ratio of the anhydride to the compound of formula (I) is from about 1:10 to about 10:1. In another embodiment, the molar feed ratio of the anhydride to the compound of formula (I) is from about 1:5 to about 5:1. In yet another embodiment, the molar feed ratio of the anhydride to the compound of formula (I) is from about 1:3 to about 3:1.

In some embodiments, the catalytic complex may be fed to the enriched product stream at a temperature from about 0° C. to about 50° C.

In some embodiments, the catalytic complex may be fed in a batch-wise manner. In one embodiment, the catalytic complex is added in a continuous manner. In another embodiment, the catalytic complex is staged across the reaction zone.

One advantage of this stable azeotrope between acetic acid and triethylamine is that the mixture can be easily distilled and recycled back into the catalytic transformation step without further purification into its individual constituents. Fresh component make-ups are used to replenish the anhydride and amine levels as a result of reacted and/or fugitive losses. In some embodiments, the catalytic complex also serves as a carrier for the liberated acid from the catalytic transformation step. In other embodiments, the build-up of byproduct acid is purged from the azeotropic complex via distillation separation and the azeotropic complex is recycled back. Overall, we find the catalytic complex management is very simple and of high utilization with minimum waste streams.

Another aspect of the disclosed process is directed to a method for making a compound of formula (V):

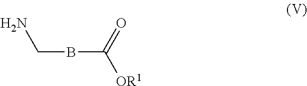

comprising
a. contacting a compound of formula (I′):

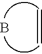

and a reagent with a medium comprising ozone to form a compound of formula (IIa′):

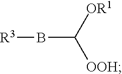

b. allowing the compound of formula (IIa′) transform to a compound of formula (IV):

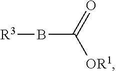

without forming an acetate side product; and
c. converting the compound of formula (IV) to the compound of formula (V), wherein B is a C₆-C₁₀ alkylene chain; and R¹ is an alkyl group, and R³ is an oxygenated functional group.

B, R¹, R³ are defined as stated above.

The compound of formula (I′) may include cyclic olefins. In some embodiments, the compound of formula (I′) is a cycloalkene having between eight and twelve carbon atoms in the molecular structure. Examples of the compound of formula (I′) include, but are not limited to, cyclohexene, cycloheptene, cyclooctene, cyclononene, cyclodecene, cycloundecene, cyclododecene, including isomers and mixtures thereof. In some embodiments, the compound of formula (I′) is cyclododecene or cyclooctene. In another embodiment, the compound of formula (I′) is a cyclooctene, including cis/trans isomers and mixtures thereof. In another further embodiment, the compound of formula (I′) is a cyclododecene, including cis/trans isomers and mixtures thereof.

In some embodiments, the compound of formula (I′) has a purity of at least 90%. In other embodiments, the compound of formula (I′) has a purity of at least 95%. In a further embodiment, the compound of formula (I′) has a purity of at least 98%. In another further embodiment, the compound of formula (I′) has a purity of at least 99%.

In some embodiments, the reagent is a C₁-C₁₀ alcohol. In other embodiments, the reagent is chosen from a class of primary, secondary, tertiary alcohols, and mixtures thereof. In some other embodiments, the reagent is chosen from methanol, ethanol, 1-propanol, 2-methyl-propan-1-ol (iso-butanol), 2-methyl-2-propanol, 1-butanol, and mixtures thereof. In yet other embodiments, the reagent is chosen from 2-propanol, 2-butanol, 3-pentanol, and mixtures thereof. In some other embodiments, the reagent is chosen from 2-methyl-2-propanol, 2-methyl-2-butanol, tert-butanol, and mixtures thereof. Higher alcohols such as butanols, etc., are preferred.

In some embodiments, the reagent is anhydrous, preferably contains less than 0.5 wt. % water, or more preferably less than 0.1 wt. % water. In other embodiments, the water content may be no more than 0.08 wt. %, preferably no more than 0.04 wt. %.

In some embodiments, the amount of the reagent may vary and generally excess reagent may be used. For purposes of the disclosed process, the molar ratio of the compound of formula (I′) to the reagent may be about 100:1 to about 1:100, preferably about 25:1 to about 1:25, and more preferably about 10:1 to about 1:10. In one embodiment, the molar ratio of the compound of formula (I′) to the reagent is about 4:1 to about 1:10. In another embodiment, the molar ratio of the compound of formula (I′) to the reagent is about 6:1 to about 1:6. In yet another embodiment, the molar ratio of the compound of formula (I′) to the reagent is about 3:1 to about 1:3.

In some embodiments, the concentration of the compound of formula (I′) in the reaction zone, by weight, may be about 0.1% to 99.9% range. In one embodiment, the compound of formula (I′) is present in the about 0.5%-25% concentration range. In other embodiments, the compound of formula (I′) is present in the about 25%-35%, in the about 35%-45%, in the about 45%-55%, in the about 55%-65%, in the about 65%-75%, in the about 75%-85%, in the about 85%-95%, or in the about 95-99.9% concentration range. In some embodiments, the compound of formula (I′) concentration range may be from about 1% to about 99%. In a further embodiment, the compound of formula (I′) concentration range may be from about 10% to about 90%. In another further embodiment, the compound of formula (I′) concentration range may be from about 25% to about 85%, preferably from about 30% to about 75%, and more preferably from about 30% to about 65%. In one embodiment, the compound of formula (I′) is from about 35% to about 60% by weight.

In some embodiments, the transformation is in the presence of a catalyst. In other embodiments, the catalyst is selected from the salts of C₁-C₆ carboxylic acids. In another embodiment, salts of alkyl amine and carboxylic acid may be used as the catalyst. In one embodiment, triethyl ammonium acetate may be used as the catalyst. In another embodiment, the catalyst system may be comprised of sodium acetate in acetic acid.

In some embodiments, the contact between the compound of formula (I′) and the reaction medium may be established using multi-phase contacting devices that are commonly known in the chemicals manufacturing industry. Examples are tower column, horizontal contactor, packed column, trickle-bed column, CSTR, tube reactor, bubble column, static mixer, jet reactor, micro-structure reactor, and variations thereof. The devices may be used alone, in sequence, in parallel or as combination of two or more. The described examples are non-limiting and those skilled in the art may appreciate all arrangement variations of such multi-phase contacting devices to achieve an efficient exchange of materials and heat which may result in acceptable product yields and quality. Whichever the arrangement may be, readers may also recognize that achieving a safe operation is the primary objective from a commercial standpoint.

In some embodiments, the contact between the compound of formula (I′) and ozone medium may be carried out in a counter-current flow mode. The counter-current flow mode means the two or more reacting phases are traveling in the opposite direction of each other. In other embodiments, the contact may occur in a co-current flow mode. The co-current flow mode means the two or more reacting phases are travelling in the same direction of each other. Other flow modes for the contact may include, but not limited to, sparged-flow, cross-flow, up-flow, down-flow, laminar-flow, turbulent-flow, thin film-flow, dispersion-flow, circulatory-flow, and combinations thereof.

In some embodiments, the contacting device may be equipped with an external or internal loop-around for efficient mixing and heat exchange. In one embodiment, the ozone medium may be introduced in the high-turbulence, loop-around section of the contacting device. In another embodiment, the ozone medium may be introduced through a distribution system across the reaction zone. In yet another embodiment, the ozone medium may be staged across the reaction zone to develop the desired spatial concentration profiles.

In one embodiment, the medium comprising ozone may be appropriately introduced to minimize the ozone entrainment in the gas-phase and to minimize aerosol formation. In another embodiment, the liquid droplets (or aerosol) from the offgas vent may be trapped and removed from the gas space. Most commonly used trapping devices include mist eliminator, aerosol coalescing section, spray section, cyclone separator, baffled serpentine flow section, and combinations thereof. The trapping section may or may not be temperature controlled. The trapped material may be returned back to the reaction zone or diverted for recovery.

In some embodiments, the reactor off-gas comprising the entrained hydrocarbons, ozone, oxygen may be adequately treated with the use of non-catalytic or catalytic thermal oxidation (TO), physical or chemical scrubbing, bio-ponds, and other known industrial abatement techniques. In another embodiment, the offgas may be fed to a co-gen facility for the fuel value recovery. In yet another embodiment, the offgas may be a useful organic food for a biological cell culture. In a further embodiment, the offgas may be an effective oxygen-rich feed for the solid-oxide fuel cell power generation system.

In some embodiments, the compound of formula (I′) is a cyclic olefin. In other embodiments, the compound of formula (I′) is selected from the group consisting of cyclooctene, cyclodecene and cyclododecene.

In some embodiments, the conversion from the compound of formula (IV) to the compound of formula (V) is conducted under a suitable reductive amination condition.

In one embodiment, the suitable reductive amination condition comprising a reductive amination agent.

Examples of suitable reductive amination agent include, but are not limited to, ammonia.

In some embodiment, the molar ratio range of ammonia and the compound of formula (IV) is from about 1:10 to about 50:1, from about 1:4 to about 20:1, or from about 1:2 to about 10:1.

Another aspect of the disclosed process is directed to a composition of matter comprising a compound of formula (IX):

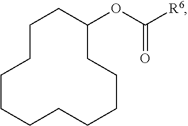

wherein R⁶ is C₄-C₂₀ alkyl or benzyl.

Another aspect of the disclosed process is directed to a composition of matter comprising a compound of formula (X):

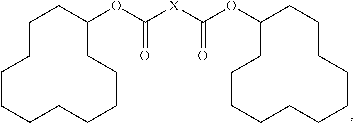

wherein X is C₁-C₁₂ alkyl, phenyl group with optional substitution, or C₁-C₄ alkyl-phenyl-C₁-C₄ alkyl.

In some embodiments, the compound of formula (IX) and/or (X) may be obtained by transforming a macrocyclic alcohol. Suitable macrocyclic alcohols may include C₆-C₂₀ cyclo-alcohols. In one embodiment, cyclododecanol (CDDA);

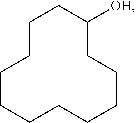 [SMILES: OC1CCCCCCCCCCC1]

may bring unique and desirable attributes to its transformed products because of its macrocyclic characteristics. The transformed products of CDDA may be used in a wide variety of applications including plasticizers, lubricants, polymer and lubricity additives, fragrance additives, and specialty solvents. The macrocyclic, transformed products may offer formulation flexibility with improved thermal stability, volatility and lubricity attributes. The lower molecular weight, high volatility products may be particularly suited in fragrance additive applications. The high molecular weight products may be useful as high boiling point specialty solvents, as processing aids or as internal lubricants for PVC plastics. Some of the products may also be useful in high temperature applications due to their high melt points.

### Process

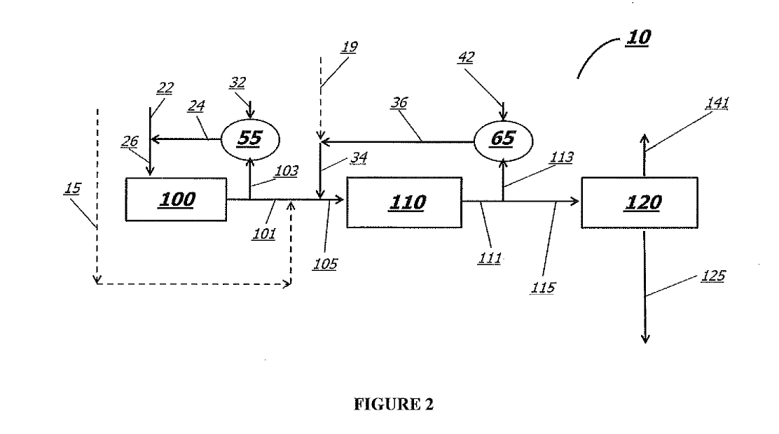

Referring more particularly to the drawing, FIG. 2 shows a non-limiting example embodiment involving the process sequence as required herein.

An embodiment 10 is given for illustrative purposes. Any of streams 15, 19 or 22 refers to a process stream comprising the compound of formula (III). These streams may optionally include one or more inert solvents for the ease of handling or otherwise. In one embodiment, stream 22 is combined with the recycle stream 24 and the mixed feed stream 26 is introduced to the hydrogenation device 100. A medium comprising dry, hydrogen is co-fed as make-up stream 32 to replenish the consumed hydrogen in unit 100. The unit 100 effluent is separated using the conventional or non-conventional techniques into the hydrogenated product stream 101, comprising the compound of formula (IV), and the hydrogen-depleted stream 103. The recycle device 55 recovers the recyclable hydrogen from stream 103 and maintains an acceptable hydrogen balance for unit 100. The compound of formula (IV) present in stream 103 may be economically recovered in unit 55 for yield improvement.

Moving forward, stream 101, comprising the compound of formula (IV), is flowed to unit 110 as the combined feed stream 105 after co-feeding stream 34. The co-feed stream 34 comprising the hydrogen, ammonia and/or ammonium medium is obtained as the recycle stream 36 from the recycle device 65. Medium comprising dry hydrogen, ammonia and/or the ammonium sub-medium is supplied to device 65 as the make-up stream 42 to replenish the consumed hydrogen and/or ammonia in unit 110. The unit 110 effluent stream 111 is separated using the conventional or non-conventional techniques into the product stream 115, comprising the compound of formula (V), and the depleted-hydrogen/ammonia stream 113. The recycle device 65 recovers the recyclable hydrogen/ammonia from stream 113 and maintains acceptable hydrogen/ammonia balances for unit 110. The compound of formula (V) present in stream 113 may be economically recovered in unit 65 for yield improvement.

The hydrogenation device 100 provides suitable hydrogenation condition for the conversion of the compound of formula (III) into the compound of formula (IV).

The unit 110 provides suitable reductive amination condition for the conversion of the compound of formula (IV) into the compound of formula (V).

In an embodiment represented in FIG. 2, the reductively aminated product stream 115, comprising the compound of formula (V), is next taken to a product recovery-purification device 120. The inert solvent, when present, is removed as a spent solvent stream 141. Stream 141 may be collected and further processed to obtain a purified solvent for partial or total recycle (not shown). The compound of formula (V) is obtained in essentially good purity from stream 125.

In a non-limiting embodiment, product recovery in device 120 may include such conventional techniques as solvent exchange, extraction, adsorption, absorption, distillation, decantation, evaporation, precipitation, saltification, crystallization, zone crystallization, membrane purification, and combinations thereof. Optionally, the inert solvent may be removed in this step via any of the conventional techniques described above such as to maintain good recovery efficiency.

In one embodiment, unit 100 may be partially or entirely bypassed and either of the streams 15 or 19 may be directly fed to unit 110 to achieve the desired unsaturation in stream 125. In another embodiment, a pre-determined portion of stream 22 may bypass unit 100 and routed (not shown) to unit 110 to obtain the desired unsaturation in stream 125.

### Analytical Methods

GC:

Analysis is conducted using an Agilent 7890A GC equipped with an FID. The GC column is an Agilent DB-1 column, 60 meter long with a diameter of 0.32 micron and a film thickness of 1.00 micron. NMP is used as the internal standard. Typically, the injection volume is 1 microliter. The injection port temperature is 250° C. with an inlet pressure of 10.0 psi. The total Helium flow is 11 ml/min with a split ratio of 10:1. The GC oven ramp program is typically; 40° C. initial temp. (no hold time); 10° C./min ramp rate; 200° C. (15 min. hold time); 10° C./min ramp rate; 275° C. final temp. (15 min. hold time). The detector is set at 275° C. with a Hydrogen flow of 40 ml/min, 400 ml/min air, 24 ml/min Helium make up. The reaction product is analyzed for various organic compounds. In addition, samples are derivatized using N,O-Bis(trimethylsilyl)trifluoroacetamide (BSTFA) in order to detect and quantify carboxylic acid type products. The GC data is used to determine the weight percent of starting material and products. The weight percent data is used to calculate conversion of starting material and the molar selectivity for various products.

HPLC:

The non-thermal sample analysis is conducted using an Agilent 1200 Series HPLC equipped with an ELSD. The HPLC column used is a Kinetex C₁₈ column having 4.6 dia. (X) 150 mm long (X) 5.00 micron film thickness. The LC samples are prepared as 1.0 mg sample in 1.0 ml butanol to yield approximately 1000 ppm sample concentration. Typically, the injection volume is 5.0 microliter. The HPLC column is maintained at 40° C. temperature and 250 bar pressure. The column flow rate is 1.0 ml/min. Acetonitrile and 0.1% formic acid are used for the linear gradient as follows:

TABLE 1

| | % | % Formic acid |
|---|---|---|
| Time (min) | Acetonitrile | [0.1%] |
| Initial | 10.0 | 90.0 |
| 9.00 | 70.0 | 30.0 |
| 9.50 | 90.0 | 10.0 |
| 17.00 | 90.0 | 10.0 |
| 17.10 | 10.0 | 90.0 |
| 20.00 | 10.0 | 10.0 |

The following Examples further demonstrate the disclosed method and its capability for use. The invention is capable of other and different embodiments, and its several details are capable of modifications in various apparent respects, without departing from the scope and spirit of the disclosed process. Accordingly, the Examples are to be regarded as illustrative in nature and not as restrictive. All percentages are by weight unless otherwise indicated.

### Example 1

In a 125cc jacketed Hastealloy C-22 autoclave (EasyMax Pressure Reactors; Mettler Toledo), 10 g of hydrocarbon liquid feed is charged together with 40 g of dry methanol at 15° C. The hydrocarbon feed is obtained by selective mono-ozonolysis of 1,5,9-cyclododecatriene (CDDT) in the presence of ozone gas and n-butanol as solvent, followed by catalytic transformation using acetic anhydride and triethylamine, followed by solvent removal, and product recovery by short-path distillation (SPD). The feed is GC-analyzed to contain 8.5 g n-butyl ester of 12-oxo-4,8-dodecadienoic acid, 0.2 g di-n-butyl ester of 4-octenedioic acid, 0.07 g n-butyl ester of 8-oxo-4-enoicoctenoic acid, 0.06 g 4,8-dodecadiene dialdehyde, 0.04 g CDDT, and 1.1 g of unidentified components. About 0.5 g of an industrially available, hydrogenation catalyst (Evonik Noblyst® P1092 Pd/C) is added to the autoclave in slightly wet form. The autoclave is sealed and pressure tested with 500 Psig nitrogen and slow stirring. Next, the headspace nitrogen is purged with 200 Psig dry hydrogen gas at 15° C., supplied from a high-pressure gas reservoir. The autoclave mixture is warmed to 20° C. while stirring at 900 RPM and the head pressure maintained at 200 Psig of hydrogen pressure. After observing an initial exotherm from 20° C. to 24.8° C. in the first 16 minutes, the mixture is maintained at 25° C. for additional 30 minutes.

The autoclave is depressurized followed by purging with nitrogen to remove residual hydrogen from the headspace. The reaction product slurry is carefully removed from the autoclave and filtered to separate the spent catalyst from the liquid. 46.6 g of the filtered product is recovered from the filtration. The overall mass balance is satisfied to 93%. The filtered reaction product is GC-analyzed to contain 8.62 g n-butyl ester of 12-oxo-dodecanoic acid, 0.2 g di-n-butyl ester of octanoic acid, less than 0.1 g of each of n-butyl ester of 8-oxo-octanoic acid, 1,12-dodecanedialdehyde, cyclododecane (CDD), and 0.9 g of other combined hydrogenated species.

The calculated conversion of n-butyl ester of 12-oxo-4,8-dodecadienoic acid to n-butyl ester of 12-oxo-dodecanoic acid is 99% molar, The feed ratios of hydrogen-to-ester product are 2.51 (moles/mole) and 0.02 (wt/wt). The corresponding yields of the product (formula (IV) compound): reactant (formula (III) compound) are 1.0 (mole/mole) and 1.02 (wt/wt).

### Example 2

Following the equipment and general procedure described in Example 1, approximately 47 g of the filtered reaction product of Example 1 is charged to the reaction autoclave at 10° C. To the autoclave 0.75 g of industrially available, reductive amination catalyst powder (W.R Grace Raney® Ni 3111) is added as slightly wet powder. The autoclave is sealed and pressure tested with 500 Psig nitrogen and slow stirring. Next, the headspace nitrogen is purged with 200 Psig dry hydrogen gas at 10° C., supplied from the high-pressure gas reservoir of Example 1. About 8 g of chilled liquid ammonia is slowly added in a stepwise manner to the autoclave, initially maintained at 10° C., while keeping the bulk temperature rise of no more than 2° C. with each addition. Upon ammonia addition is complete, the autoclave temperature is gradually increased to 50° C. and the hydrogen pressure in the autoclave headspace is maintained at 700 Psig for 58 minutes with stirring at 900 RPM. The autoclave is then slowly depressurized at 50° C. The reaction mixture is cooled to room temperature and the dilute slurry product with spent catalyst is carefully poured out of the autoclave.

The reaction product is filtered to remove the solid catalyst and 52.2 g of the filtered liquid is recovered for analysis. The filtered product is LC-analyzed to contain 8.8 g of n-butyl ester of 12-amino-dodecanoic acid, 0.33 g n-butyl ester of 12-oxo-dodecanoic acid as unconverted feed, and 0.9 g of other unidentified species.

The calculated conversion of n-butyl ester of 12-oxo-dodecanoic acid to n-butyl ester of 12-amino-dodecanoic acid is 96% molar. The feed ratios of hydrogen-to-ester feed are 3.6 (moles/mole) and 0.03 (wt/wt). The corresponding ratios of ammonia-to-ester feed are 14.6 (moles/mole) and 0.92 (wt/wt). The corresponding yields of the product (formula (V) compound): reactant (formula (IV) compound) are 1.0 (mole/mole) and 1.01 (wt/wt).

### Example 3

The so-obtained product of Example 2, n-butyl ester of 12-amino-dodecanoic acid (formula (V) compound) is recovered and purified by a conventional low-temperature crystallization technique followed by several re-crystallizations, solvent washing and vacuum drying. The solvents used are from a class of alcohols, such as methanol, iso-propyl alcohol, n-butanol.

### Example 4

Following the equipment and general procedure of Example 1, 15 g of hydrocarbon liquid feed is charged together with 35 g of dry methanol at 15° C. The hydrocarbon feed is obtained from the CDDT mono-ozonolysis process as. The feed is GC-analyzed to contain 12.7 g n-butyl ester of 12-oxo-4,8-dodecadienoic acid, 0.3 g di-n-butyl ester of 4-octenedioic acid, 0.1 g n-butyl ester of 8-oxo-4-enoicoctenoic acid, 0.1 g 4,8-dodecadiene dialdehyde, 0.06 g CDDT, and 1.8 g of unidentified components. About 0.5 g of an industrially available, hydrogenation catalyst ((Evonik Noblyst® P 1086 Pd/C) is added to the autoclave in slightly wet form. The operating procedure of Example 1 is followed for the reaction conditions of 200 Psig hydrogen pressure and 25° C. temperature for 30 minutes at 900 RPM stirring. After observing an initial exotherin from 20° C. to 23.3° C., the mixture is maintained at 25° C. for additional 30 minutes.

Following the Example 1 procedure, 47.7 g of the filtered reaction product is recovered from the autoclave. The overall mass balance is satisfied to 95%. The filtered reaction product is GC-analyzed to contain 13.9 g n-butyl ester of 12-oxo-dodecanoic acid, 0.3 g di-n-butyl ester of octanoic acid, 0.16 g n-butyl ester of 8-oxo-octanoic acid, 0.22 g 1,12-dodecanedialdehyde, and 0.13 g cyclododecane (CDD).

The calculated conversion of n-butyl ester of 12-oxo-4,8-dodecadienoic acid to n-butyl ester of 12-oxo-dodecanoic acid is 100% molar. The feed ratios of hydrogen-to-ester product are 1.34 (moles/mole) and 0.01 (wt/wt). The corresponding yields of the product (formula (IV) compound): reactant (formula (III) compound) are 1.08 (mole/mole) and 1.1 (wt/wt).

### Example 5

Following the equipment and general procedure of Example 2, 47.2 g of the filtered reaction product of Example 4 is charged to the reaction autoclave at 5° C. To the autoclave 0.75 g of industrially available, reductive amination catalyst powder (W.R Grace Raney® Ni 3111) is added as slightly wet powder. The autoclave is sealed and pressure tested with 500 Psig nitrogen and slow stirring. Next, the headspace nitrogen is purged with 200 Psig dry hydrogen gas at 10° C., supplied from the high-pressure gas reservoir of Example 1. Lowering the reactor pressure to 100 Psig and stirring to 90 RPM, 3 g of chilled liquid ammonia is slowly added in a stepwise manner to the autoclave, initially maintained at 10° C., while keeping the bulk temperature rise of no more than 2° C. with each addition. Upon ammonia addition is completed, the operating procedure of Example 2 is followed for the reaction conditions of 700 Psig hydrogen pressure and 50° C. temperature for 120 minutes at 900 RPM stirring. About 49 g of the reaction product is recovered using the procedure of Example 2. The reaction product is filtered to remove the solid catalyst. The filtered product is LC-analyzed to contain 12.1 g of n-butyl ester of 12-aminododecanoic acid, 0.21 g n-butyl ester of 12-oxo-dodecanoic acid as unconverted feed, and 0.17 g of n-butyl ester of 12-oxo-4,8-dodecadienoic acid.

The calculated conversion of n-butyl ester of 12-oxo-dodecanoic acid to n-butyl ester of 12-amino-dodecanoic acid is 98% molar. The feed ratios of hydrogen-to-ester feed are 2.4 (moles/mole) and 0.02 (wt/wt). The corresponding ratios of ammonia-to-ester feed are 3.5 (moles/mole) and 0.22 (wt/wt). The corresponding yields of product (formula (V) compound): reactant (formula (IV) compound) are 0.88 (mole/mole) and 0.88 (wt/wt).

### Example 6

A 500 mL jacketed, round-bottom glass reactor is charged with 35 g (0.32 mole) 1,5-cycicooctadiene and 65.0 g (0.88 mole) 1-butanol. A flow of 21% O₂ in argon is fed to the ozone generator followed by flowing to an ozone monitor. A flow of 2 Liters/min (LPM) is set on the ozone generator that flowed to the ozone monitor. A steady state (20-30 min) concentration of 33.0 g ozone/m³ in argon is measured continuously on the monitor. After ˜15 min at steady state, the feed ozone in argon is diverted to the reactor. The jacketed glass reactor containing, a mechanical stirrer, a tube for the ozone addition, an exit gas fitting and a fourth port for addition of reagents and sampling with a thermocouple is maintained at minus 5° C. The coolant from the circulating bath (13 Liters, 15 LPM) is circulated through the vessel. The gas is flowed through the reactor followed by passing through a Dry Ice cold trap followed by a scrubber containing 66 g tetradecane. The ozone monitor continually measured the ozone concentration fed during the run. The run time is 141 min. The ozone generator is then turned off and nitrogen is then passed into the reactor for 5 min to remove any residual ozone.

When the reaction is complete, un-reacted 1-butanol is removed under high vacuum at <50° C. (max) and ˜462-472 millitorr. The reactor is warmed to 25° C. followed by the addition of 32 g (0.31 mole) acetic anhydride and run for 15 min. About 12 g (0.12 mole) triethylamine is next added while maintaining the mixture temperature below 25° C. After the complete addition the reaction is run for 120 minutes. The conversion of 1,5-cyclooctadiene is 81% (92% accounted for with the remaining lost in the off gas). Selectivity to 8-oxo-octa-4-enoic acid butyl ester is 84.1% along with selectivities of 7.5%, 6.1% and 3.1% to 4-oxo-succinic acid butyl ester, dibutyl succinate and 1,8-octadial, respectively.

### Example 7

The recovered product of Example 6 comprising 8-oxo-octa-4-enoic acid butyl ester is catalytically hydrogenated using equipment and procedures described in Example 1 to obtain the butyl ester of 8-oxo-octanoic acid in quantitative yield. Thus hydrogenated C₈ butyl ester is catalytically aminated in the presence of hydrogen/ammonia and using equipment and procedures described in Example 2. The reductively aminated product is analyzed to be n-butyl ester of 8-amino-octanoic acid in quantitative yield.

### Example 8

A continuous-flow, ½ inch outside diameter (0.43 inch inside diameter) by 20-inch long 316 stainless steel reactor tube is used. The unit is operated in the up-flow mode and the gas and liquid phases are co-current, i.e., flow in the same direction from bottom to top.

About 10 g of Evonik Noblyst® 1010 (Palladium on silica support) catalyst is loaded into the above tube reactor. The reactor flows are established using a 10 wt % solution of n-butyl ester of 12-oxo-4,8-dodecadienoic acid in iso-propyl alcohol [IPA] as carrier solvent and pressurized hydrogen. The reaction zone is maintained at 500 Psig pressure and 25° C. bed temperature via active jacket heat transfer. The hydrocarbon ester feed, is obtained according to equipment and procedures similar to Example 1, i.e., by selective mono-ozonolysis of CDDT in the presence of ozone gas and n-butanol at partial conversion, followed by catalytic transformation using acetic anhydride and triethylamine, followed by solvent removal, and product recovery by thermal methods.

Hydrogenation is continued in the flow reactor at 0.52 cc/min liquid feed via the feed pump and about 35.1 std. cc/min hydrogen gas feed via the high-pressure reservoir. These flows result in the Weight Hourly Space Velocity [WHSV] of about 0.25 g/hr undiluted reactant feed per g catalyst. The liquid product discharged upon gas disengagement and depressurization is collected and analyzed on a calibrated GC.

The hydrogenated product mainly comprises of n-butyl ester of 12-oxo-dodecanoic acid in almost quantitative yield. The calculated conversion of n-butyl ester of 12-oxo-4,8-dodecadienoic acid to n-butyl ester of 12-oxo-dodecanoic acid is more than 95% molar.

### Example 9

The flow reactor assembly as described in Example 8 is used for reductive amination using inter-changeable flow reactor tube; this time in the presence of anhydrous ammonia and hydrogen. For reductive amination, an identical flow reactor tube is packed with molybdenum-promoted Raney® Ni fixed bed catalyst (W.R Grace Raney® Ni 5831). The anhydrous, liquid ammonia is fed from the liquid feed tank equipped with a dip tube.

The reactor flows are established using a liquid feed comprising n-butyl ester of 12-oxo-dodecanoic acid in iso-propyl alcohol [IPA] as carrier solvent and pressurized hydrogen. The reaction zone is maintained at 700 Psig pressure and 60° C. bed temperature via active jacket heat transfer. Reductive amination is performed at about 8.91 ml/min liquid feed, about 0.28 ml/min anhydrous ammonia and about 316 std. cc/min hydrogen gas feed. At these feed rates the molar excess hydrogen and ammonia are about 2.5 and 3.0 times the stoichiometric amounts, respectively. The liquid product discharged after excess H₂/NH₃ gas disengagement and depressurization is collected and analyzed on a calibrated HPLC. The product mainly comprises of n-butyl ester of 12-amino-dodecanoic acid in almost quantitative yield.

### Example 10

Preparation of 12-oxo-dodecanoic acid, n-butyl ester from cyclododecene ozonolysis: A 500 ml jacketed round-bottom glass reactor is fitted with a dry ice condenser, mechanical stirrer, stainless steel feed tube for sub-surface ozone gas addition and a fourth port for addition of reagents, sampling and thermocouple connection. A dry gas mixture containing 21% oxygen in argon is fed to an ozone generator (Pacific Ozone). The exit gas from the ozone generator is flowed through an ozone monitor (Teledyne Instruments) for 30 min to observe stable ozone concentration in the feed gas. The reaction temperature is maintained at a desired target via jacketed cooling. The exit gas containing residual oxygen and argon are passed through a dry ice cold trap to recover any low-boiling components. Upon reaction time completion, dry nitrogen is passed into the reactor for 30 min to displace any residual ozone and oxygen and the vessel is warmed to room temperature.

Cyclododecene, used in this example, is obtained from Alfa Aesar (A Johnson Matthey Company). The purity of Cyclododecene is 97% by weight. A steady concentration of 26.2 g ozone/m³ is sparged sub-surface into the reaction vessel containing a liquid mixture of 45 g (0.27 moles) of cyclododecene and 60 g (0.81 moles) of n-butyl alcohol that is dried over the molecular sieves. The total ozone fed is 15 g at the 0.10 g/min feed rate, which is equivalent to the molar feed ratio of 1.15 ozone/CDDT. The reaction is carried out at 5° C. bulk temperature for 150 minutes. When the reaction is complete, a cooled liquid mixture of 55 g acetic anhydride and 8.25 g triethylamine is added to the well-agitated reaction intermediate. The reaction exotherms, observed during these additions, are managed by active jacketed cooling to ensure the temperature is maintained at or below 10° C. The reaction mixture is allowed to reach room temperature and stirred for additional 30 minutes for completion. 168 g of one-phase liquid reaction product is recovered. The final GC analysis indicates complete conversion of cyclododecene and n-butyl ester of 12-oxo-dodecanoic acid is the major product.

### Example 11

The recovered product of Example 10 comprising n-butyl ester of 12-oxo-dodecanoic acid is reductively aminated in the presence of hydrogen/ammonia and using the equipment and procedures described in Example 2. The reductively aminated product is analyzed to be n-butyl ester of 12-amino-dodecanoic acid in quantitative yield.

### Example 12

Preparation of 8-amino-octanoic acid, n-butyl ester from cyclooctene ozonolysis: A 95 wt % minimum purity cis-cyclooctene is purchased from Sigma Aldrich. Employing the reactor and procedure similar to Example 10, ozonolysis of cyclooctene in the presence of dry n-butanol followed by its transformation with acetic anhydride and triethylamine produces n-butyl ester of 8-oxo-octanoic acid in high selectivity. The cyclooctene conversion is complete within 150 minutes and at 5° C. reaction temperature.

The recovered product of cyclooctene ozonolysis transformation is reductively aminated using equipment and procedures described in Example 2. The product obtained is n-butyl ester of 8-amino-octanoic acid. The calculated conversion of n-butyl ester of 8-oxo-octanoic acid to n-butyl ester of 8-amino-octanoic acid is nearly 100% molar.

### Example 13

Approximately 25 g of hydrocarbon liquid feed comprising n-butyl ester of 12-oxo-4,8-dodecadienoic acid in 35 g of 1-butanol is charged to the autoclave assembly described in Example 1. About 0.7 g of hydrogenation catalyst (Evonik Noblyst® P1092 Pd/C) is added to the autoclave in slightly wet form. The autoclave mixture is hydrogenated for 75 minutes, using the procedure described in Example 1, and at 25° C. mixture temperature and 200 Psig head pressure. The recovered product after filtration is analyzed to be 98.9% n-butyl ester of 12-oxo-dodecanoic acid on a solvent-free basis.

### Example 14

Following the equipment and general procedure described in Example 2, approximately 37 g of the filtered product of Example 13 is reductively aminated for 6 hours at 60° C. mixture temperature and 600 Psig head pressure using 30 g ammonia (as aqueous ammonium hydroxide) in the presence of 3.4 g of W.R Grace Raney® Ni 3111 catalyst. The organic portion of the filtered reactor effluent is analyzed to be 89.9% n-butyl ester of 12-amino-dodecanoic acid.

### Example 15

Using the continuous-flow tube reactor packed with about 10 g of Johnson Matthey (P-978; 1% Pd/C) hydrogenation catalyst and general procedure of Example 8, a hydrocarbon feed comprising 10 wt % n-butyl ester of 12-oxo-4,8-dodecadienoic acid in iso-propyl alcohol [IPA] is hydrogenated at 25° C. and 750 Psig pressure. The liquid feed rate is about 4.15 cc/min and the hydrogen feed rate is about 280.7 std. cc/min. These flows result in the Weight Hourly Space Velocity [WHSV] of about 2.0 g/hr undiluted reactant feed per g catalyst. The liquid product discharged upon gas disengagement and depressurization is collected and analyzed on a calibrated LC. The LC-detected composition of n-butyl ester of 12-oxo-dodecanoic acid in the recovered product after 4 hours of continuous operation is 96.2% on a solvent-free basis.

### Example 16-25

Cyclododecanol (CDDA; CAS No. 1724-39-6) is obtained from INVISTA S.a.r.l. and other starting materials and reagents obtained from Aldrich Chemical. Products are analyzed, where appropriate, by gas chromatography on a DB-1701 column and HP 6890 instrument. Melting points are determined using a Fisher Thomas hot stage melting point apparatus. Viscosity is measured using a Brookfield LVF viscometer.

Example 19 is a preparation of an ester using CDDA and dodecanedioic acid (DDDA). In this example, a solution of dodecanedioyl chloride (21.4 g, 0.08 mole) in 50 mL toluene is drop wise added over 2 h to a stirred solution of cyclododecanol (29.5 g, 0.16 mole) and pyridine (12.6 g, 0.16 mole) in 100 mL toluene under nitrogen atmosphere at 75° C. The reaction mixture is stirred at 75° C. for an additional 1.5 h, cooled to room temperature, and filtered through a fitted glass funnel to remove pyridine hydrochloride. The hydrochloride salt is washed with 75 mL toluene and both filtrates are combined, then vacuum stripped on a rotary evaporator to remove toluene. Bis(cyclododecyl)dodecanedioate is obtained (36.9 g, 0.066 mole, 82% yield) as a viscous, honey color oil which slowly crystallizes to a brownish solid.

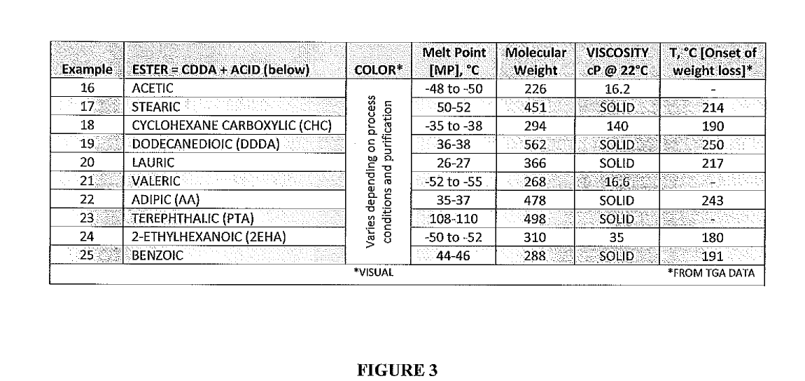

The preparation of esters from CDDA is made according to the published literature methods, for example, U.S. Pat. No. 3,440,894 and N. Sonntag, Chem. Rev., 52, 237-416 (1953). Other CDDA esters, prepared using the above procedure and corresponding acids, are shown in FIG. 3 along with their measured properties.

The wear performance data for the prepared CDDA esters, according to Examples 16-25, is obtained using a fuel lubricity test, CEC-F-06-A-96, which is designed specifically to evaluate the lubricating properties of diesel fuel to ensure satisfactory operation of fuel injection equipment. The test involves a 6 mm steel ball that oscillates against a stationary flat steel disk with the point of contact submerged in a pool of lubricant fluid. Test parameters that are kept constant during the test are: 60° C. temperature, 200 g load, 1000 μm stroke length, 50 Hz Oscillation frequency and 75 minutes of test duration. Film thickness and friction coefficient are measured continuously during the test. The wear scar on the ball is measured after the test is complete.

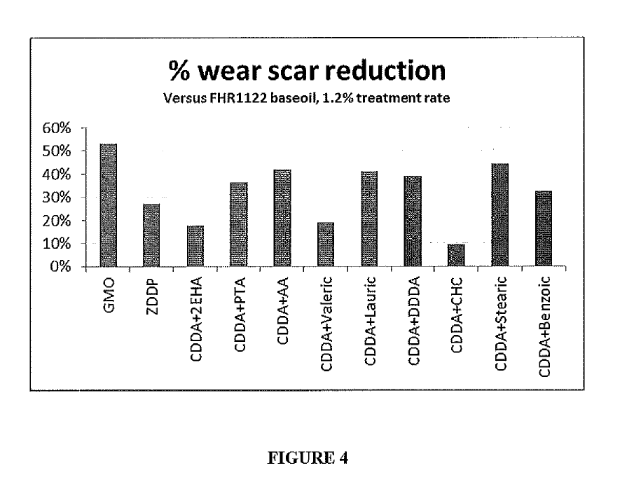

FIG. 4 gives a representation of the wear scar reduction performance of the CDDA esters, prepared according to Examples 16 through 25. In FIG. 4, GMO is glycerol monooleate, ZDDP is zinc dialkyldithiophosphate and FHR 1122 is Flint Hills paraffinic hydrocracked base oil. The treatment rate is with 1.2% by weight of the corresponding ester in the base oil.

### Example 26

1,5,9-cyclododecatriene (CDDT) is used as received from INVISTA™ Specialty Intermediates. Table 2 depicts a typical composition.

TABLE 2
Typical 1,5,9-cyclododecatriene composition
Typical Composition, wt %

| | |
|---|---|
| 1,5,9-Cyclododecatriene | >99 |
| Tert-butyl catechol | 30-50 ppm |
| Isomers | |
| cis, trans, trans | 98 |
| trans, trans, trans | 1.5 |
| cis, cis, trans | 0.3 |
| cis, cis, cis | 0.1 |

Employing the reactor and procedure described in Example 10, a 2 liter/min total gas flow, containing a steady concentration of 26.3 g ozone/m³ is sparged sub-surface into the reaction vessel for 60 minutes containing a liquid mixture of 35 g (0.22 moles) of CDDT and 20 g of n-butyl alcohol that is dried over the molecular sieves. The total ozone fed is 3.15 g at the 0.053 g/min feed rate, which is equivalent to the molar feed ratio of 0.3 ozone/CDDT. The reaction is carried out at 5° C. bulk temperature. When the reaction is complete, a cooled liquid mixture of 20 g acetic anhydride and 2.5 g triethylamine is added to the reaction intermediate via pump at an average feed rate of 2.81 g/min with 650 RPM stirring of the reaction mixture. The reaction exotherms, observed during these additions, are managed by active jacketed cooling to ensure the temperature is maintained at or below 10° C. The reaction mixture is allowed to reach room temperature and stirred for additional 30 minutes for completion.

73 g of one-phase liquid reaction product is recovered. The final GC analysis indicates 39.5% CDDT conversion. The GC-analyzed, solvent-free, product weight distribution of the C₁₂ components is: 12.2 g n-butyl ester of 12-oxo-dodeca-4,8-dieneoic acid, 0.5 g dodeca-4,8-diene-1,12-dialdehyde and 0.05 g 12-oxo-dodeca-4,8-dieneoic acid. The non-selective products are: 0.1 g unsaturated C₈ dialdehyde, 0.3 g other unsaturated C₈ components and 0.1 g of combined C₄ impurities. 21.2 g of CDDT fed remaines unconverted in this example. The normalized molar selectivity of the reacted CDDT is 90.5% for n-butyl ester of 12-oxo-dodeca-4,8-dieneoic acid, 5.1% for dodeca-4,8-diene-1,12-dialdehyde, 0.5% for 12-oxo-dodeca-4,8-dieneoic acid, 0.85% for unsaturated C₈ dialdehyde and 2.5% for the combined other unsaturated C₈ and saturated C₄ impurities.

The above product is purified to obtain n-butyl ester of 12-oxo-dodeca-4,8-dieneoic acid in high purity (+90%). Following the equipment and general procedures described in Example 1 (hydrogenation condition) and Example 2 (reductive amination condition), the n-butyl ester of 12-oxo-dodeca-4,8-dieneoic acid is quantitatively converted to n-butyl ester of 12-oxo-dodecanoic acid and then to n-butyl ester of 12-amino-dodecanoic acid.

What is claimed is:

1. A method for making a compound of formula (V): 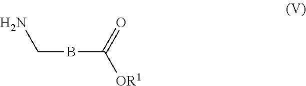
comprising converting a compound of formula (III): 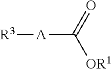
to the compound of formula (V), wherein A is a C₆-C₁₀ alkene group having at least one carbon-carbon double bond, B is a C₆-C₁₀ alkylene chain; and R¹ is an alkyl group, and R³ is an oxygenated functional group.

2. The method of claim 1, wherein the compound of formula (III) is first converted to a compound of formula (IV):

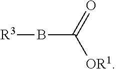

3. The method of claim 1, wherein the compound of formula (III) is first converted to a compound of formula (VI):

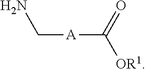

4. The method of claim 1, wherein R¹ is C₁-C₅ alkyl.

5. The method of claim 1, wherein R³ is an aldehyde.

6. The method of claim 2, wherein the conversion to the compound of formula (IV) is conducted under a suitable hydrogenation condition.

7. The method of claim 6, wherein the suitable hydrogenation condition comprising a hydrogenation agent.

8. The method of claim 7, wherein the hydrogenation agent is hydrogen.

9. The method of claim 8, wherein the molar ratio range of hydrogen and the compound of formula (III) is from about 1:10 to about 50:1.

10. The method of claim 9, wherein the molar ratio range of hydrogen and the compound of formula (III) is from about 1:5 to about 20:1.

11. The method of claim 2, wherein the conversion from the compound of formula (IV) to the compound of formula (V) is conducted under a suitable reductive amination condition.

12. The method of claim 11, wherein the suitable reductive amination condition comprising a reductive amination agent.

13. The method of claim 12, wherein the reductive amination agent is ammonia.

14. The method of claim 13, wherein the molar ratio range of ammonia and the compound of formula (IV) is from about 1:10 to about 50:1.

15. The method of claim 14, wherein the molar ratio range of ammonia and the compound of formula (IV) is from about 1:4 to about 20:1.

16. A method for making a compound of formula (V): 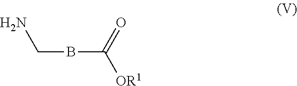
comprising
a. contacting a compound of formula (I): 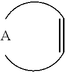
and a reagent with a medium comprising ozone to form a compound of formula (IIa): 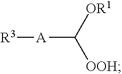
b. allowing the compound of formula (IIa) transform to a compound of formula (III): 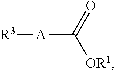
without forming an acetate side product; and
c. converting the compound of formula (III) to the compound of formula (V), wherein A is a C₆-C₁₀ alkene group having at least one carbon-carbon double bond, B is a C₆-C₁₀ alkylene chain; and R¹ is an alkyl group, and R³ is an oxygenated functional group.

17. The method of claim 16, wherein the compound of formula (III) is first converted to a compound of formula (IV):

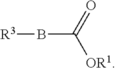

18. The method of claim 16, wherein the compound of formula (III) is first converted to a compound of formula (VI):

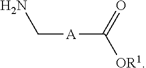

19. A composition of matter comprising a compound of formula (IX): 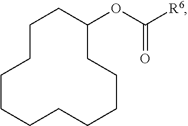
wherein R⁶ is C₄-C₂₀ alkyl or benzyl.

20. A composition of matter comprising a compound of formula (X): 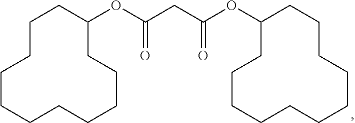
wherein X is C₁-C₁₂ alkyl, phenyl group with optional substitution, or C₁-C₄ alkyl-phenyl-C₁-C₄ alkyl.
